(19) United States
(12) Patent Application Publication
(10) Pub. No.: US 2025/0135116 A1
(43) Pub. Date: May 1, 2025

(54) Syringe Handheld Safety Apparatus

(71) Applicant: Nikolaos Pahis, Wethersfield, CT (US)

(72) Inventor: Nikolaos Pahis, Wethersfield, CT (US)

(21) Appl. No.: 18/383,592

(22) Filed: Oct. 25, 2023

(51) Int. Cl. A61M 5/24

(52) U.S. Cl. CPC A61M 5/2422

(57) ABSTRACT

A novel syringe handheld safety apparatus design to accept a vial contain a medical fluid and a medical syringe operatable to imbibe a fluid residing in said vial comprising: an interchangeable vial carrier formed to support said medical vial, an interchangeable syringe carrier formed to support said medical syringe and an interchangeable syringe plugger struck limiter defining the limit of fluid volume stored in said medical syringe. The syringe handheld safety apparatus, the vial, and the syringe, are cooperative associated with each other in order to imbibe said medical fluid from said vail and store it in the syringe chamber in a safe, precise, and efficient manner.

### BACKGROUND OF THE INVENTION

This invention relates to a syringe handheld safety apparatus that is employed to imbibe a medical liquid fluid (namely liquid fluid) from a medical vial (namely vial) with a conventional medical syringe (namely syringe) and store it in a syringe fluid chamber which is formed in between a reciprocate syringe plunger and a syringe barrel, and more specifically, to a syringe handheld safety apparatus comprising of an interchangeable vial delegated carrier (namely vial carrier), an interchangeable member or, alternatively, an interchangeable assembly syringe delegated carrier (namely syringe carrier) and an interchangeable syringe plunger struck limiter (namely struck limiter).

Namely, there is provided a syringe handheld safety apparatus comprising of an interchangeable vial carrier formed to support a vial containing a liquid fluid therein, an interchangeable syringe carrier and an interchangeable struck limiter employed to limit the struck length of said syringe plunger thereby controlling the fluid volume stored in the syringe chamber. Said syringe handheld safety apparatus is operatable to assists said syringe, initially, to imbibe air in order to fill a syringe chamber, thereafter thrust said vail carrier until the syringe needle fully penetrates the vial seal and the syringe needle opening is fully exposed to liquid fluid in order to inject said imbibed air therein and subsequently imbibe a liquid fluid therefrom to be stored syringe chamber and used for a medical treatment. It has long been known in the prior art to provide a syringe filling system embodying different types of construction, such as, a syringe filling system that employs a human hand to partially retrieve a syringe plunger from a syringe barrel in order to imbibe a liquid fluid from a vial through a syringe needle that is mounted on said syringe barrel employed to penetrate through a seal of said vial in order to access the liquid fluid residing therein.

It is also known in the prior art to provide in such syringe filling apparatus embodying different types of construction that are capable of providing different kinds of performance. A prior art form of a syringe filling system such a syringe filling system that employs a human hand to imbibe liquid medicine from a medicine bottle is that illustrated in U.S. Pat. No. 4,219,055. Which issued in August 1980. U.S. Pat. No. 4,219,055, is directed to syringe filling aid comprising a body having a bottle holding means thereon preferably in the form of resilient jaws for securing a medicine bottle to the body, a hilt-engaging portion attached to the body and spaced in one direction from the bottle holding means and adapted to engage that side of the hilt which is opposite to the needle of the tube so as to limit movement of the tube away from the bottle when the needle is in the bottle, is the main combination of the invention. The afford mentioned U.S. Pat. No. 4,219,055, further includes a telescopic extension with a slot for adjustment, restrained thereon with a bolt assembly, employed to control the traveling of the afford mentioned syringe plunger.

Another prior art form of syringe filling apparatus is a syringe guide and vial holder suitable for use to fill a syringe is that illustrated in U.S. Pat. No. 5,894,870, which issued on Apr. 20, 1999. U.S. Pat. No. 5,894,870 is directed to a syringe guide and vial holder facilitates the insertion of a syringe into a medicine vial and the subsequent withdrawal of medicine from a vial into syringe. The syringe guide and the vial holder addressed herein employ the human hands to manipulate and position a medicine vial relative to syringe in order to extract the medicine contained in said vial.

Another prior art form of syringe filling apparatus is a syringe guide and vial holder suitable for use to fill a syringe is that illustrated in U.S. Pat. No. 6,497,697, which issued on Dec. 24, 2002. U.S. Pat. No. 6,497,697 is directed to a syringe guide and vial holder, employed human hands to facilitates the insertion of a syringe needle into a medicine vial and the subsequently withdraw of medicine from a vial and store it into syringe.

Although methods for imbibing a liquid fluid from a vial such embodying a guide, a syringe and a medicine vial, which have been constructed in accordance with the teaching of the prior art such as those set forth, for example, in the aforementioned patents, are capable of providing adequate performance in transferring a liquid fluid medicine from a medical vial into syringe, a need has nevertheless been evidenced for improvements in the area of efficiency, accuracy and safety to be made thereto,

One such improvement, resides in the desire to provide a syringe handheld safety apparatus, which is operative for the purpose of accurately imbibing with a syringe a liquid fluid from a vial to be used for a medical treatment and prevent medical overdose.

Another such improvement, resides in the desire to provide a syringe handheld safety apparatus, which is operative for the purpose to accurately imbibing with a syringe a liquid fluid from a vial to be used for a medical treatment and prevent a medical underdose.

Still another such improvement, resides in the desire to provide a syringe handheld safety apparatus, which is operative for the purpose of safely imbibing with a syringe a liquid fluid from a vial to be used for a medical treatment and avoid syringe needle health personal injuries.

Another such improvement, resides in the desire to provide a syringe handheld safety apparatus, which is operative for the purpose of efficiently imbibing with a syringe a liquid fluid from a vial to reduce medical waste.

Still such improvement, resides in the desire to be able to provide a syringe handheld safety apparatus, which is operative to interchange a vial carrier of said apparatus for the proposes of accommodating a variety of vial sizes.

Another such improvement, resides in the desire to be able to provide a syringe handheld safety apparatus, which is operative to interchange a syringe carrier of said apparatus for the purposes of accommodating a variety of syringe sizes.

Still such improvement, resides in the desire to provide a syringe handheld safety apparatus, which is operative to interchange a syringe struck limiter of said apparatus for the purpose of imbibing with a syringe a liquid fluid from a vial and accurately filling a syringe chamber.

Anther such improvement, resides in the desire to provide a syringe handheld safety apparatus, which is operative to provide an interchangeably and adjustable syringe plunger struck limiter of said apparatus for the purpose imbibing with a syringe a liquid fluid from a vial and accurately filling a syringe chamber.

Still another such improvement, resides in the desire to be able to provide a syringe handheld safety apparatus, which is operative to offset the centerline of a vial carrier relative to syringe centerline for the purpose of preventing vial seal degradation during multi-dose vial and multi-needle penetrations.

### SUMMARY OF THE INVENTION

In accordance with the present invention, there is provided a syringe handheld safety apparatus comprising of an interchangeable vial carrier, an interchangeable syringe carrier and an interchangeable struck limiter. Said vial carrier employed to assist a vial to precisely interact with a syringe needle for a limited vial seal penetration in order to access and fully utilize a medical fluid residing therein. Said syringe carrier employed to carry a syringe and support a vial carrier and a struck limiter. Said struck limiter employed to limit the struck length of a syringe plunger in order to accurately imbibe a fluid volume to be stored in a syringe chamber.

More specifically, the syringe handheld safety apparatus that is particularly suited to safely fill a syringe chamber, accurately fill a syringe chamber to prevent a medical overdose, accurately fill a syringe chamber for preventing a medical underdose, to minimize medical fluid waste and all in efficient manner. Said syringe handheld safety apparatus comprises a vail carrier employed to support a sealed vial which contains a liquid fluid therein, a syringe carrier employed to support a syringe and a struck limiter employed to limit the traveling of syringe plunger thereby defining the volume of the liquid fluid stored in a syringe chamber.

Moreover, the syringe handheld safety apparatus, the vial and the syringe, are cooperatively associated one with another such that a vail carrier is able to undergo a linear reciprocate motion between two predefined positions to accomplish a syringe needle vial seal limited penetration in order to access a liquid fluid residing therein or, alternatively being in a fixed position and said syringe plunger is able to undergo a linear reciprocate motion between two predefined positions, initially to imbibe air in order to fill the syringe chamber, thereafter injected the imbibed air in said vial in order to create an pressure equilibrium and finally imbibe a liquid fluid therefrom and store it in the syringe chamber to be used for a medical treatment.

### BRIEF DESCRIPTION OF THE DRAWINGS

FIG. 1 is a 3D side elevational view of a conventional syringe in a traditional position, which is employed to imbibe a liquid fluid from a conventional vial and which is constructed in accordance with the teaching of the prior art.

FIG. 2 is a side elevational view of a first embodiment of a syringe handheld safety apparatus illustrated in an inactive mode, which embodies a vial, a syringe, an interchangeable vial carrier, an interchangeable syringe carrier and a multi-position fluid volume struck limiter, employed to imbibe a liquid fluid from said vial and store it in said syringe chamber, constructed in accordance with the present invention;

FIG. 2A is an end view of a first embodiment of the syringe handheld safety apparatus, depicted in FIG. 2 taken substantially along the line 2A-2A in FIG. 2, constructed in accordance with the present invention;

FIG. 2B is a cross-sectional view of a first embodiment of the syringe handheld safety apparatus, depicted in FIG. 2 taken substantially along the line 2B-2B in FIG. 2, constructed in accordance with the present invention;

FIG. 2C is a cross-sectional view of a first embodiment of the syringe handheld safety apparatus, depicted in FIG. 2 taken substantially along the line 2C-2C in FIG. 2, constructed in accordance with the present invention;

FIG. 2D is a cross-sectional view of a first embodiment of the syringe handheld safety apparatus, depicted in FIG. 2 taken substantially along the line 2D-2D in FIG. 2, constructed in accordance with the present invention;

FIG. 2E is a cross-sectional view of a first embodiment of the syringe handheld safety apparatus, depicted in FIG. 2 taken substantially along the line 2E-2E in FIG. 2, constructed in accordance with the present invention;

FIG. 2F is a cross-sectional view of a first embodiment of the syringe handheld safety apparatus, depicted in FIG. 2 taken substantially along the line 2F-2F in FIG. 2, constructed in accordance with the present invention;

FIG. 2G is a cross-sectional view of a first embodiment of the syringe handheld safety apparatus, depicted in FIG. 2 taken substantially along the line 2G-2G in FIG. 2, constructed in accordance with the present invention;

FIG. 2H is a cross-sectional view of a first embodiment of the syringe handheld safety apparatus, depicted in FIG. 2 taken substantially along the line 2H-2H in FIG. 2, constructed in accordance with the present invention;

FIG. 3 is an exploded side elevational view of a second embodiment of a syringe handheld safety apparatus, which embodies an interchangeable vial carrier, an interchangeable syringe carrier and a multi-position interchangeable fluid volume struck limiter, constructed in accordance with the present invention;

FIG. 3A is a sectional side elevational view of a second embodiment of a syringe handheld safety apparatus illustrated in an active mode, which embodies a vial, a syringe, an interchangeable vial carrier, an interchangeable syringe carrier and a multi-position interchangeable fluid volume struck limiter, employed to imbibe a liquid fluid from said vial and store it in said syringe chamber, constructed in accordance with the present invention;

FIG. 3B is a sectional side elevational view of a second embodiment of a syringe handheld safety apparatus illustrated in an inactive mode, which embodies a vial, a syringe, an interchangeable vial carrier, an interchangeable syringe carrier and a multi-position interchangeable fluid volume struck limiter, employed to imbibe a liquid fluid from said vial and store it in said syringe chamber, constructed in accordance with the present invention;

FIG. 3C is an end view of a second embodiment of the syringe handheld safety apparatus, depicted in FIG. 3B taken substantially along the line 3C-3C in FIG. 3B, constructed in accordance with the present invention;

FIG. 3D is a cross-sectional view of a second embodiment of the syringe handheld safety apparatus, depicted in FIG. 3B taken substantially along the line 3D-3D in FIG. 3B, constructed in accordance with the present invention;

FIG. 3E is a cross-sectional view of a second embodiment of the syringe handheld safety apparatus, depicted in FIG. 3B taken substantially along the line 3E-3E in FIG. 3B, constructed in accordance with the present invention;

FIG. 3F is a cross-sectional view of a second embodiment of the syringe handheld safety apparatus, depicted in FIG. 3B taken substantially along the line 3F-3F in FIG. 3B, constructed in accordance with the present invention;

FIG. 3G is a cross-sectional view of a second embodiment of the syringe handheld safety apparatus, depicted in FIG. 3B taken substantially along the line 3G-3G in FIG. 3B, constructed in accordance with the present invention;

FIG. 3H is a cross-sectional view of a second embodiment of the syringe handheld safety apparatus, depicted in FIG. 3B taken substantially along the line 3H-3H in FIG. 3B, constructed in accordance with the present invention;

FIG. 3I is a cross-sectional view of a second embodiment of the syringe handheld safety apparatus, depicted in FIG. 3B taken substantially along the line 3I-3I in FIG. 3B, constructed in accordance with the present invention;

FIG. 3J is a cross-sectional view of a second embodiment of the syringe handheld safety apparatus, depicted in FIG. 3B taken substantially along the line 3J-3J in FIG. 3B, constructed in accordance with the present invention;

FIG. 3K is a cross-sectional view of a second embodiment of the syringe handheld safety apparatus, depicted in FIG. 3B taken substantially along the line 3K-3K in FIG. 3B, constructed in accordance with the present invention;

FIG. 4 is a side elevational view partially in section of a third embodiment of a syringe handheld safety, which embodies a vial, a syringe, an interchangeable vial carrier, an interchangeable syringe carrier including an actuator and an interchangeable fluid volume struck limiter, employed to imbibe a liquid fluid from said vial and store it in said syringe chamber, constructed in accordance with the present invention;

FIG. 4A is a side elevational view partially in section of a third embodiment of a syringe handheld safety apparatus illustrated in an inactive mode, which embodies a vial, a syringe, an interchangeable vial carrier, an interchangeable syringe carrier including an actuator and an interchangeable fluid volume struck limiter, employed to imbibe a liquid fluid from said vial and store it in said syringe chamber, constructed in accordance with the present invention;

FIG. 4B is a cross-sectional view of a third embodiment of the syringe handheld safety apparatus, depicted in FIG. 4 taken substantially along the line 4B-4B in FIG. 4, constructed in accordance with the present invention;

FIG. 4C is a cross-sectional view of a third embodiment of the syringe handheld safety apparatus, depicted in FIG. 4 taken substantially along the line 4C-4C in FIG. 4, constructed in accordance with the present invention;

FIG. 4D is a cross-sectional view of a third embodiment of the syringe handheld safety apparatus, depicted in FIG. 4 taken substantially along the line 4D-4D in FIG. 4, constructed in accordance with the present invention;

FIG. 4E is a cross-sectional view of a third embodiment of the syringe handheld safety apparatus, depicted in FIG. 4 taken substantially along the line 4E-4E in FIG. 4, constructed in accordance with the present invention;

FIG. 4F is a cross-sectional view of a third embodiment of the syringe handheld safety apparatus, depicted in FIG. 4 taken substantially along the line 4F-4F in FIG. 4, constructed in accordance with the present invention;

FIG. 4G is a cross-sectional view of a third embodiment of the syringe handheld safety apparatus, depicted in FIG. 4 taken substantially along the line 4G-4G in FIG. 4, constructed in accordance with the present invention;

### DESCRIPTION OF THE PREFERRED EMBODIMENTS

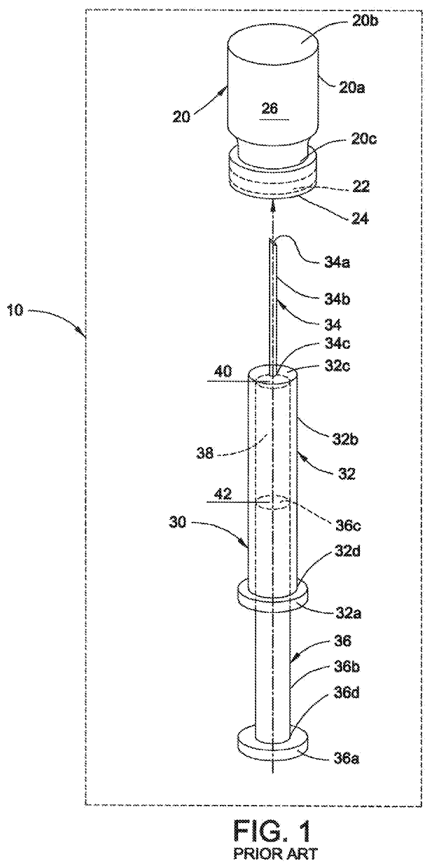

Referring to the FIG. 1 of the drawing, there is depicted therein a prior art of a conventional syringe and vial combination generally designated by the numeral 10, embodying conventional vial generally designated by the numeral 20 and a conventional syringe generally designated by the numeral 30, manually employed to imbibe a liquid fluid 26 resided in said vial 20. Said vial 20 is defined by a cylindrical portion 20a having a first end sealed end 20b and a second end namely mouth opened end 20c (not shown) suitable formed to accept conventional fluid seal means 22 restrained thereon with conventional seal retention means 24. Syringe 30 comprises, a stationery syringe barrel 32, on which is mounted rigidly a syringe needle 34 and a movable syringe plunger 36. Syringe barrel 32 is defined by a cylindrical portion 32b, a sealed end 32c that includes an opening (not shown) formed to accept saleably the first end 34c of syringe needle 34, a second end 32d on which is attached fixedly an external ring 32a provided with an opening (not shown) to accept said syringe plunger 36 for a sealable reciprocate movement therein. Syringe plunger 36 is defined by a cylindrical body 36b having a first sealed end 36c and a second end 36d on which is attached fixedly an external ring 36a. Syringe needle 34 is a cylindrical tube 34b having said first opened end 34c mounted sealably on said syringe barrel portion 32c and a second end 34a being beveled and sharp, suitable to penetrate said fluid sealing means 22 of vial 20 for the purposes of injecting air in said vial 20 and imbibing a liquid fluid 26 therefrom and store it in the syringe chamber 38.

A description will now follow herein the method employed to imbibing a liquid fluid 26 from the vial 20 and store in into syringe chamber 38. The method requires the cooperation of both human hands, (not shown), one hand to hold the vial 20 and the other hand to operate the syringe 30. As a first step, the plunger end 36c is retrieved from the first position 40 to second assigned position 42 in order to fill the syringe chamber 38 with air. Thereafter, the syringe 30 thrust against the vial 20 until the syringe needle end 34a penetrate the seal 22 and the opening fully exposed to liquid fluid 36 residing therein. At this point, the plunger 36 thrusted from the second position 42 in to first position 40 to fully inject the air from the syringe chamber 38 into the vial 20 in order to create a pressure equilibrium. Once the air fully injected in the vial 20, the plunger 36 is retrieved from the first position 40 in to the second assigned position 42 to fill the syringe chamber 38 with the liquid fluid 26. At this point the syringe 30 now can be retrieve to be used for a medical treatment.

The above conventional imbibe process, requires lengthy time, hand stability, and special attention to accuracy to in order to avoid unintended medical events. Events such as, excessive syringe needle penetration into vial leading into medical fluid loose, syringe needle misalignment which can lead to personnel injures resulting to infections with legal implications. Inaccurate fluid volume stored in the syringe chamber that can lead to underdose or underdose resulting to lose of life and legal implications.

The present patent application will at best will attempt to addressed the above mentioned unintended medical events.

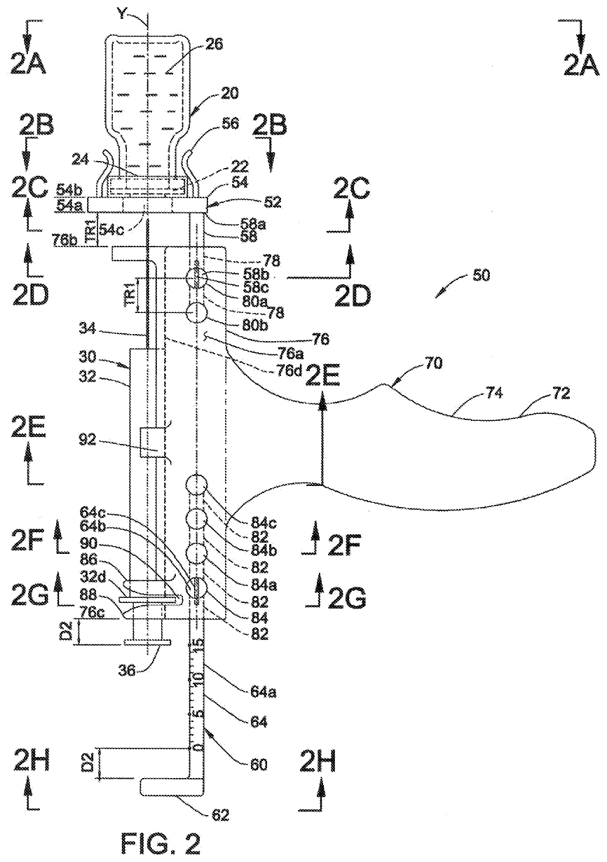

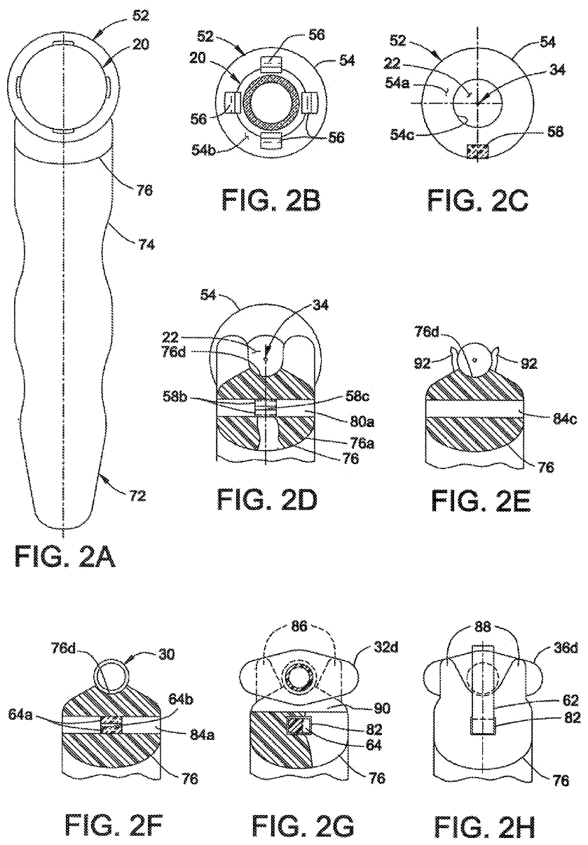

Reference now will be had to figures FIG. 2, FIG. 2A, FIG. 2B, FIG. 2C, FIG. 2D, FIG. 2E, FIG. 2F, FIG. 2G and FIG. 2H of the drawing. There is depicted therein a first embodiment of a syringe handheld safety apparatus, generally designated by the numeral 50, which embodies a vial, generally designated by the numeral 20, supported on a multi-position interchangeable vial delegated carrier, generally designated by the numeral 52, a multi-position interchangeable fluid volume delegated struck limiter, generally designated by the numeral 60, a syringe, generally designated by the numeral 30, supported on an interchangeable syringe delegated carrier, general designated by the numeral 70, constructed in accordance with the present invention.

The multi-position interchangeable vial carrier 52 shown herein partially in section comprising; a flat base 54, defined by a flat surface 54a perpendicular to an axis Y suitably formed to interact with flat surface 76b of said syringe carrier 70, a flat surface 54b parallel to surface 54a suitably formed to interface with the mouth of vial 20, a through opening 54c being in line with axis Y suitable formed to allow syringe needle 34 to pass in order to penetrate the vial seal 24 and access a liquid fluid 26 residing in the vial 20, multiple flexible vial gripping means 56 fixedly attached and equally spaced flat surface 54b extending therefrom so as to suitably restrain removably said vial 20. Member 52 further includes a parallel to axis Y nonrotation proof support/restraining means such as rectangular in section portion 58, fixedly attached with one end 58a on flat surface 54a and a second end forming a cylindrical portion 58b having a diameter larger that of rectangular section 58, divided with a slot 58c essential for constrict during a slidable insertion in a nonrotation proof such as rectangular opening 78 and restrained selectively through cylindrical restraining openings 80a and 80b on stationary member 70.

Reference will be hand now to multi-position struck limiter 60. Struck limiter 60 is a L shape member comprises of a first portion 62 employed to limit the struck length of syringe plunger 36, and a parallel to axis Y nonrotation proof support/restraining means such as rectangular in section portion 64 fixedly attached with the first end 64a on portion 62 with second end forming a cylindrical portion 64b with diameter larger that of rectangular section 64, divided with a slot 64c essential for constrict during a slidable insertion in the nonrotation proof rectangular opening 82 and restrained selectively on stationary member 70 through cylindrical restraining openings 84, 84a, 84b and 84c. Struck limiter 60 further includes engraved markings such as for example 0, 5, 10 and 15 corresponding to distances between the cylindrical opening 84, 84a, 84b and 84c of syringe carrier 70, which in turn reflects the markings and liquid fluid volume stored in the syringe chamber, while the distance D2 herein reflects the inactive distance in between the stationary member 70 and syringe plunger 36 of syringe handheld safety apparatus 50.

Continuing with reference to syringe handheld safety apparatus 50 and more particularly the syringe carrier 70. Syringe carrier 70 comprises a member 72 defined by a portion 74 suitably formed for handling by a human hand, a semicylindrical longitudinal portion 76 suitable formed to support removably said syringe 30, slidably and removably vial carrier 52 and struck limiter 60. Said longitudinal portion 76 further defined by the outer surface 76a, the perpendicular to axis Y flat end surfaces 76b and 76c, semicylindrical surface 76d with center axis Y suitable formed to accept the syringe barrel 32, a first parallel to axis Y nonrotation proof such as rectangular opening 78 intersected by two perpendicular to axis Y cylindrical restraining openings 80a and 80b suitably formed to support through portion 58 and restrain selectively through cylindrical portion 58b said vial carrier 52 on syringe carrier 70, a second parallel to axis Y nonrotation proof such as rectangular opening 82 intersected by perpendicular to axis Y multiple equally spaced cylindrical restraining openings 84, 84a, 84b and 84c suitable positioned to selectively support and restrain the member 60 through cylindrical portion 64a on syringe carrier 70, two pairs of syringe barrel stop 86 and 88 separated by a space 90 suitably positioned to accept the syringe barrel ring 36d employed to prevent the syringe 30 from an axial movement and one or more pairs of syringe barrel restraining means 92 employed to restrain removably said syringe 30 on syringe carrier 70, with TR1 indicating the traveling distance of vial carrier 52 in order for syringe needle 34 to access the liquid fluid 26 residing in vial 20.

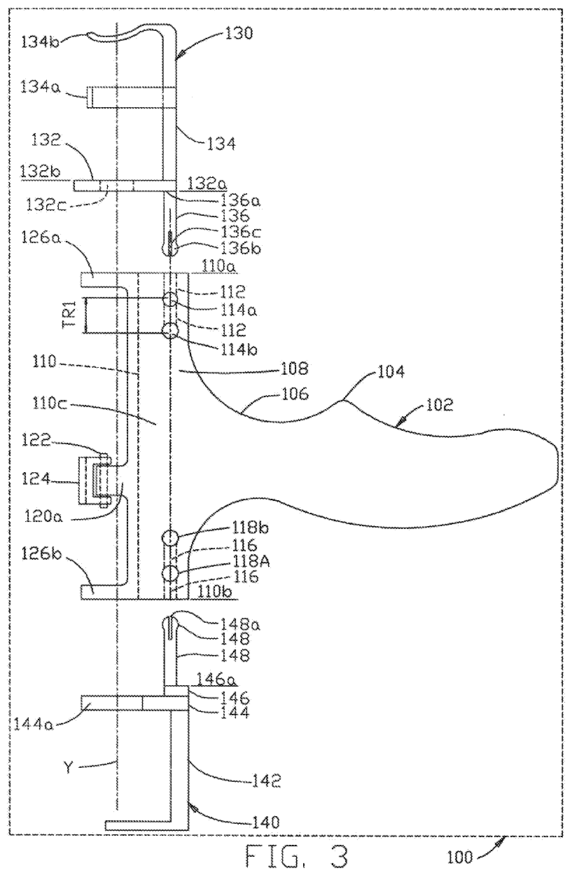

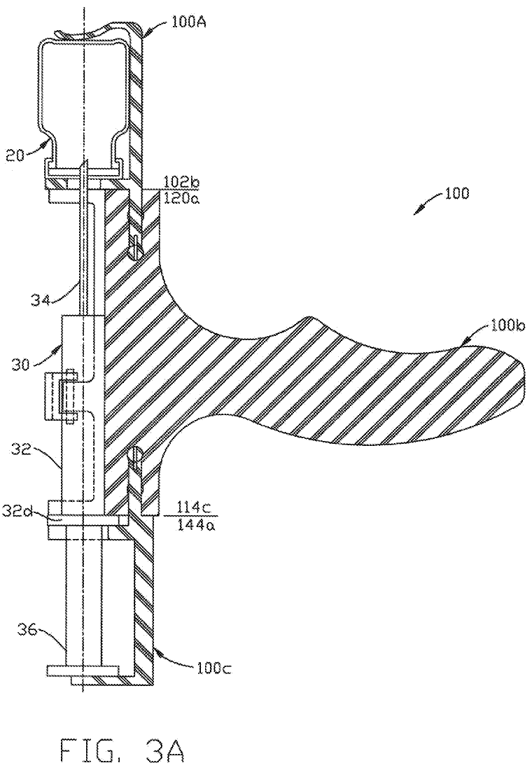

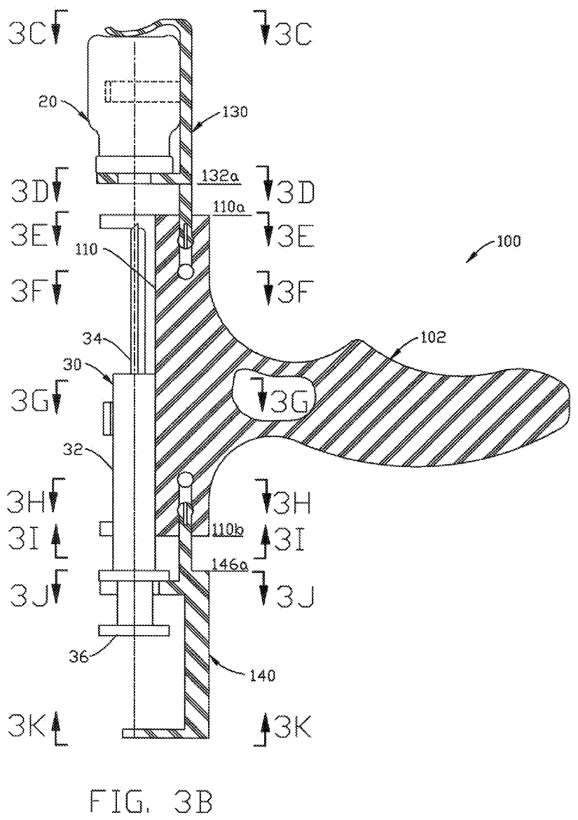

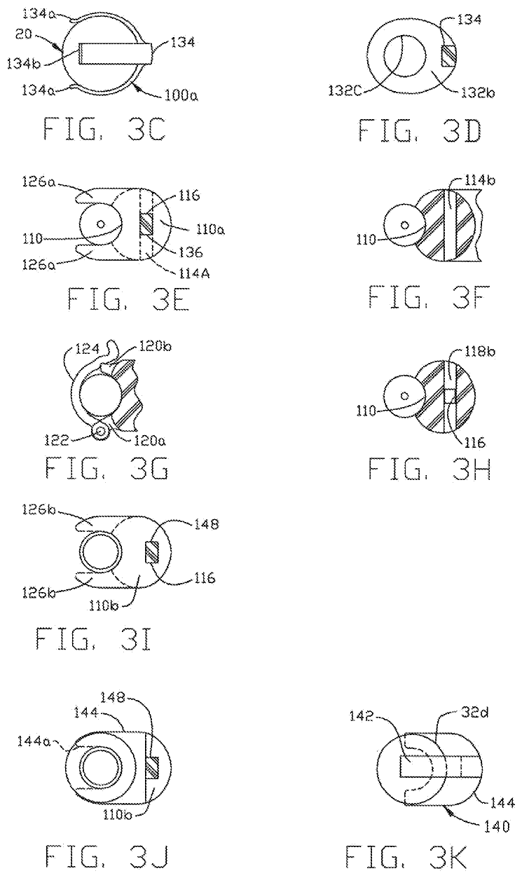

Reference now will be had to figures FIG. 3, FIG. 3A, FIG. 3B, FIG. 3C, FIG. 3D, FIG. 3E, FIG. 3F, FIG. 3G, FIG. 3H, FIG. 3J and FIG. 3K of the drawing. There is depicted therein an second embodiment of a syringe handheld safety apparatus, generally designated by the numeral 100, which embodies a syringe, generally designated by the numeral 30, supported on an interchangeable syringe delegated carrier assembly, generally designated 102, a vial, generally designated by the numeral 20, supported on a multi-position interchangeable vial delegated carrier, generally designated by the numeral 130 and a multi-position interchangeable fluid volume delegated struck limiter, generally designated by the numeral 140, constructed in accordance with the present invention.

Reference will be hand to syringe carrier assembly 102 of handheld safety apparatus 100. Syringe carrier assembly 102 comprises a member 104, a syringe restraining member 120 and shaft 122 employed to support pivotally said syringe restrain member 120 on member 102. Member 104 includes a portion 106 suitably formed for handling by a human hand, a semicylindrical longitudinal portion 108 along axis Y provided with means to support slidably and removably a syringe 30, a vial delegated carrier 130 and a struck limiter 140. Longitudinal portion 108 further includes, an inner semicylindrical surface 110 with centerline the axis Y suitable formed to accept syringe barrel 32, an outer surface 110c, two perpendicular to axis Y flat end surfaces 110a and 110b, a parallel to axis Y nonrotation proof such as rectangular opening 112 suitably formed to support slidably and removably said vial carrier 130 intersected by two perpendicular to axis Y cylindrical restraining opening 114a, 114b spaced at a distance denoted RT1, a parallel to axis Y nonrotation proof such as rectangular opening 116 suitably formed to support slidably and removably said struck limiter 140 intersected by two perpendicular to axis Y cylindrical restraining opening 118a and 118b suitably formed to restrain selectively said struck limiter 140, a portion 120a employed to support pivotally with shaft 122a syringe restraining member 124 and a portion 120b formed to interacting with syringe restraining member 124 in a luck and unluck manner, and two extended balance pairs 126a and 126b.

Continuing with the description of the multi-position interchangeable vial carrier 130. Vial carrier 130 comprising a flat base 132 defined by a perpendicular to axis Y flat surface 132a, suitably formed to interface with flat surface 110a of syringe carrier 102, a flat surface 132b parallel to surface 132a suitably formed to interface with the mouth of vial 20, an through opening 132c suitable positioned to allow syringe needle 34 to go through in order to access a liquid fluid 26 residing in said vial 20, a portion 134 being parallel to axis Y fixedly attached on flat surface 132b extended therefrom to support flexible vial restraining means 134a and 134b. Member 130 further includes a parallel to axis Y nonrotation proof support/restraining means such as portion 136 which is rectangular in section to prevent rotation, fixedly attached with the first end 136a on the flat surface 132a of base 132 and second end forming a cylindrical restraining portion 136b with diameter larger than the cross section of the rectangular portion 136 divided with a slot 136c essential for constrict during a insertion in the nonrotation proof such as rectangular opening 112 of syringe carrier member 104 and selectively restrained in the cylindrical openings 114a and 114b.

Reference will be hand now to multi-position interchangeable struck limiter 140. Struck limiter 140 comprises of a first L shape portion 142 employed to limit the struck length of syringe plunger 36 attached on a second portion 144 which includes a opening 144a employed to retrain the syringe barrel 32 on syringe carrier 102, a third portion portion 146 with thickness equal to the thickness of syringe barrel ring 32d fixedly attached to portion 144 and a fourth portion 148 nonrotation proof support/restraining means such as rectangular in section to prevent rotation during the insertion in the nonrotation proof such as rectangular opening 116 of syringe carrier 102, attached with one end on portion 146 and a second end forming a slotted cylinder 148a with slot 148b being essential for constriction during the insertion in the nonrotation proof such as rectangular opening 116 of syringe carrier 102 and restrained thereon through cylindrical openings 118a and 118b

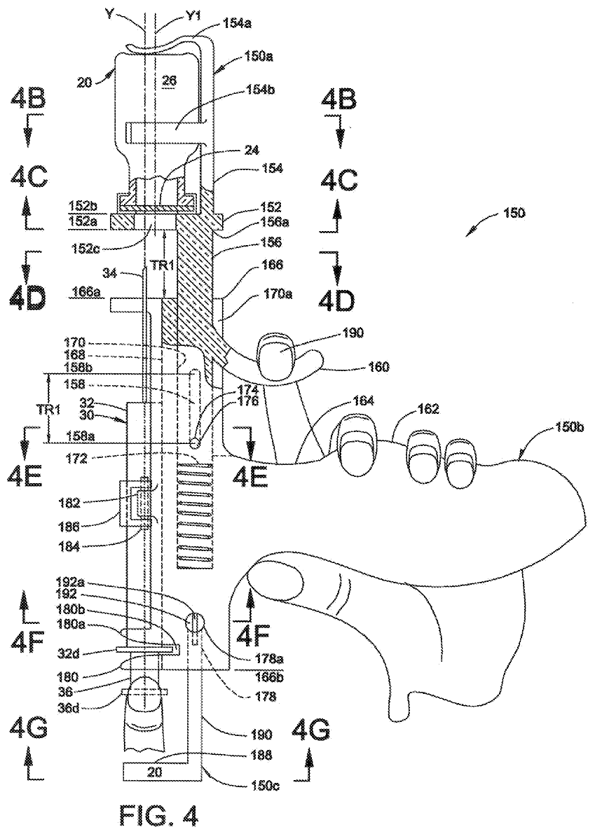

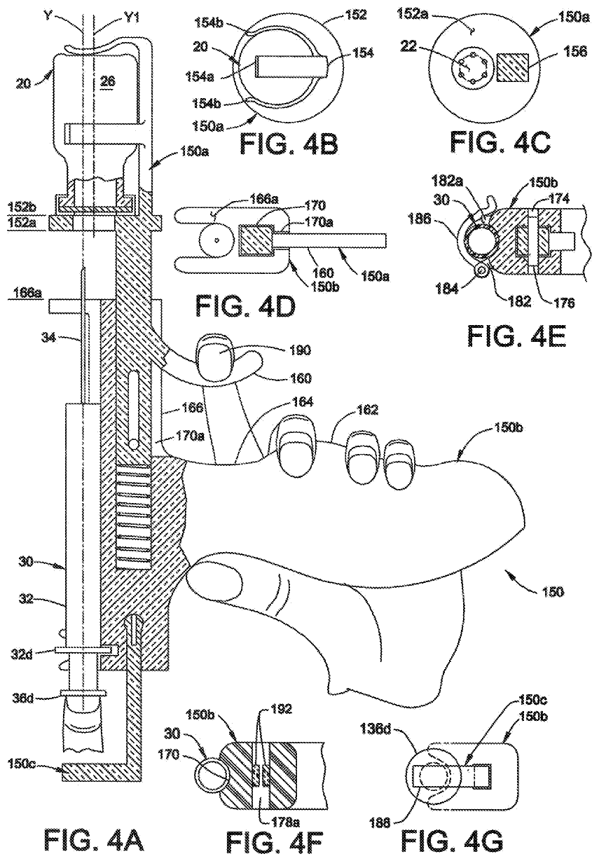

Reference now will be had in the drawing and more particularly to FIG. 4, FIG. 4A, FIG. 4B, FIG. 4C, FIG. 4D, FIG. 4E, FIG. 4F and FIG. 4G thereof. There is depicted therein a fourth embodiment of a syringe handheld safety apparatus, generally designated by the numeral 150, which embodies a vial with center axis Y1 generally designated by the numeral 20, supported on an interchangeable vial delegated carrier, generally designated by the numeral 150a, a syringe with center axis Y generally designated by the numeral 30 supported on an interchangeable syringe delegated carrier generally designated by the numeral 150b and an interchangeable struck delegated limiter generally designated by the numeral 150c, constructed in accordance with the present invention.

The interchangeable vial carrier 150a shown herein partially in section comprising of; a flat portion 152 defined by a flat surface 152a being perpendicular to axis Y and Y1 suitably formed to interact with flat surface 166a of syringe carrier assembly 150b, a flat surface 152b which is parallel to surface 152a suitably formed to interface with vial 20, a through opening 152c with center in line with axis Y1 suitable positioned to allow syringe needle 34 to penetrate the vial seal 24 and access a liquid fluid 26 residing in the vial 20, a parallel to axis Y1 portion 154 being fixedly attached on flat surface 152b extended therefrom to support flexible vial restraining means 154a and 154b employed to restrain removably said vial 20, and a parallel to Y1 axis nonrotation proof fixedly attached on flat surface 152a support portion 156 on which is attached a finger trigger portion 160 interacting with retraction means such as spring 172. Said nonrotation proof portion 156 further includes a parallel to Y1 axis stadium like opening 158 suitable for restraining movably with restraining means 176 said vial carrier 150a on said syringe carrier 150b. Said stadium like opening 158 comprise by a straight portion denoted as TR1 defining the maximum movement of vial carrier 150a relative to syringe carrier 150b connected to two semi-cylindrical end portions with centers 158a and 158b.

Continuing with reference to syringe handheld safety apparatus 150 and more particularly to syringe carrier assembly 150b. Syringe carrier 150b comprise of a member 162 defined by a portion 164 suitably formed for handling by a human hand and a semicylindrical longitudinal portion 166. Semicylindrical longitudinal portion 166 includes a first perpendicular to Y axis flat surfaces 166a suitable formed to interact with flat surface 152a of vial carrier 150a, a second perpendicular to Y axis flat surface 166b, a parallel to Y axis nonrotation proof opening 170 suitable formed to accept slidably said vial carrier support means 156, a parallel to Y axis side opening 170a leading to opening 170 suitable formed to allow for a free movement of said finger trigger 160, vial carrier retraction spring means 172, a through opening 174 suitable to accept vial carrier retention means 176, a parallel to Y axis semicylindrical surface 168 formed to accept syringe barrel 32, a nonrotation proof such a rectangular opening 178 suitable formed to support struck limiter 150c and cylindrical opening 178a suitable formed to restrain said struck limiter 150c on said syringe carrier 150b.

Continue with reference the syringe carrier 150b. Said syringe carrier further includes syringe barrel positioners 180 and 180a separated by a space 180b suitable to accept the syringe barrel ring 32d employed to prevent said syringe 30 from moving axially, a portion 182 employed to support pivotally with shaft 184 syringe restraining member 186 and a portion 182a positioned opposite to portion 182 designed to interacting with syringe restraining member 186 in a lock and unlock manner.

Reference will be hand now to syringe plunger delegated struck limiter 150c. Struck limiter 150c is a L shape member comprising of first portion 188 positioned perpendicular to Y axis employed to limit the struck length of syringe plunger 36 and on a second parallel to Y axis nonrotation proof such as rectangular in section support portion 190 suitable to prevent rotation during a sladuble insertion in a nonrotation proof opening 178 of syringe carrier 150b and a cylindrical portion 192 with slot 192a being essential for constriction during the insertion nonrotation proof opening 178 employed to restrain rigidly said struck limiter 150c on said syringe carrier 150b through cylindrical opening 178a. syringe handheld safety apparatus In order to limit the exchange of said interchangeable vial carrier 150a, a vial insert (not shown) can be employed to accommodate the a variety of vial sizes

Continue with reference with the operation of the syringe handheld safety apparatus 150. For this, a preselected vail carrier 150a, syringe carrier 150b and struck limiter 150c must be assembled and the vial 20 and syringe 30 must be in place.

Said vial carrier 150a supported in mounted relation on said interchangeable syringe carrier 150b so as to be selectively movable in a power pull manner and in power retraction manner to imbibe and inject air and imbibe fluid. Said interchangeable vial carrier 150a being movable in power pull manner between an inactive syringe needle disengaging mode when the center of rod 174 is in line with the center 158a of the semi-cylindrical opening of stadium like opening 158 and an active air inject and fluid imbibe syringe needle engaging mode when the center of rod 174 is in line with the center 158a of the semi-cylindrical opening of stadium like opening 158. Said interchangeable vial carrier 150a being movable in the power pull manner between an inactive syringe needle disengaging mode and an active syringe needle engaging mode by virtue of a dynamic pull force imparted on said interchangeable vial carrier 150a. Said dynamic pull engaging force being imparted to interchangeable vial carrier being derived from a dynamic pull impartible by a human finger. Said interchangeable vial carrier 150a being movable in the power retraction manner between an active syringe needle engaging mode and an inactive syringe needle disengaging mode by virtue of a dynamic thrust force. Said dynamic thrust force imparted on said interchangeable vial carrier 150a being derived from a dynamic thrust impartible by a compression spring, said interchangeable vial carrier being movable in the power retraction manner in an inactive syringe needle disengaging mode thereby disposed for a repeat movement. At this point, the syringe is filled, removed and ready be used for a medical treatment.

While several embodiments of the invention have been shown, it will be appreciated that still other modifications thereof, some of which have been alluded hereinabove, may have readily made thereto by those skilled in the art such for example vial and syringe carrier combination or syringe and struck limiter combination. I thereof, intend by the appended claims to cover the modifications alluded to herein as well as all the other modifications which fall within the true spirit and scope of the invention.

What is claimed is:

1. A syringe handheld safety apparatus assembly comprising:
a. An interchangeable syringe carrier supporting for movement an interchangeable vial carrier through nonrotation proof vial carrier support and restraining means, an interchangeable struck limiter through nonrotation proof struck limiter support and restraining means and a syringe;
b. said interchangeable vial carrier supported in mounted relation on said interchangeable syringe carrier so as to be selectively movable in a power thrust manner and in power retraction manner, said interchangeable vial carrier being movable in power thrust manner between an inactive syringe needle disengaging mode and in an active syringe needle engaging mode by virtue of a force imparted on the interchangeable vial carrier, said force being imparted to interchangeable vial carrier being derived from a dynamic thrust impartible by a human hand, said interchangeable vial carrier being movable in the power retraction manner in between an active syringe needle engaging mode and an inactive syringe needle disengaging mode, said interchangeable vial carrier being movable in the power retraction manner in an inactive syringe needle disengaging mode thereby disposed for a repeat movement;
c. said interchangeable struck limiter being supported in mounted relation on said interchangeable syringe carrier, positioned relative to syringe plunger so as to define a syringe plunger struck length, said syringe plunger struck length being associable to a fluid volume stored in said fluid chamber formed in between a syringe barrel and a syringe plunger of said syringe.

2. A syringe handheld safety apparatus as set forth in claim 1 where an interchangeable vial carrier comprises a flat portion which include an opening, flexible vial gripping means, nonrotation proof vial carrier support means and vial carrier restraining means. Said flat portion has a first flat surface and a second flat surface, said first flat surface faces said vial and a second flat surface faces said syringe carrier, said multiple flexible vial gripping means are fixedly attached on said first flat surface of said flat portion, said vial carrier support means are fixedly attached on said second flat surface of said flat portion said restraining means are fixedly attached on said vial carrier support means.

3. A syringe handheld safety apparatus as set forth in claim 1 where an interchangeable vial carrier comprised of a flat portion which include an opening, flexible vial restraining means, nonrotation proof vial carrier support means and vial carrier restraining means. Said flat portion has a first flat surface and a second flat surface, said first flat surface faces said vial and a second flat surface faces said syringe carrier, said flexible vial restraining means are fixedly attached on said first flat surface of said flat portion, said vial carrier support means are fixedly attached on said second flat surface of said flat portion, said vial carrier restraining means are fixedly attached on said vial carrier support means.

4. A syringe handheld safety apparatus as set forth in claim 1 where said interchangeable vial carrier constructed as to be movably between two predefined position and selectively restrained rigidly on said interchangeable syringe carrier.

5. A syringe handheld safety apparatus as set forth in claim 1 where said interchangeable vial carrier fixedly supported on said interchangeable syringe carrier.

6. A syringe handheld safety apparatus as set forth in claim 1 where said interchangeable struck limiter constructed as to be supported movably and selectively restrained rigidly on a predefined position on said interchangeable syringe carrier.

7. A syringe handheld safety apparatus as set forth in claim 1 where said interchangeable struck limiter constructed as to be movably between two predefined position and selectively restrained rigidly on said interchangeable syringe carrier.

8. A syringe handheld safety apparatus as set forth in claim 1 where said interchangeable struck limiter fixedly supported on said interchangeable syringe carrier.

9. A syringe handheld safety apparatus as set forth in claim 1 where said interchangeable struck limiter slidably supported on said interchangeable syringe carrier, constructed as to be movable between multiply predefined positions and selectively rigidly restrained therein.

10. A syringe handheld safety apparatus comprising:
a. An interchangeable syringe carrier supporting for movement an interchangeable vial carrier through nonrotation proof vial carrier support and restraining means, an interchangeable struck limiter through nonrotation proof struck limiter support and restraining means, retraction means, retention means and a syringe;
b. said interchangeable vial carrier supported in mounted relation on said interchangeable syringe carrier so as to be selectively movable in a power thrust manner and in power retraction manner, said interchangeable vial carrier being movable in power thrust manner between an inactive syringe needle disengaging mode and in an active syringe needle engaging mode by virtue of a force imparted on the interchangeable vial carrier, said power thrust force being imparted to interchangeable vial carrier being derived from a dynamic pull impartible by a human finger, said interchangeable vial carrier being movable in power retraction manner between an active syringe needle engaging mode and in an inactive syringe needle disengaging mode by virtue of a force imparted on the interchangeable vial carrier, said power retraction force being imparted to interchangeable vial carrier being derived from a dynamic thrust impartible by a compassion spring, said interchangeable vial carrier being movable in power retraction manner between an active syringe needle engaging mode and in an inactive syringe needle disengaging mode thereby disposed for a repeat movement;
c. said interchangeable struck limiter being supported in mounted relation on said interchangeable syringe carrier, positioned relative to syringe plunger so as to define a syringe plunger retraction struck length, said syringe plunger retraction struck length being associable to a fluid volume stored in a fluid chamber formed in between the syringe barrel and the syringe plunger of said syringe.

11. A syringe handheld safety apparatus as set forth in claim 1 where an interchangeable vial carrier comprised of a flat portion including an opening, flexible vial restraining means, nonrotation proof vial carrier support means, vial carrier finger thrust means and vial carrier stadium like opening retention means. Said flat portion has a first flat surface and a second flat surface, said first flat surface faces said vial, said a second flat surface faces said syringe carrier, said flexible vial restraining means are fixedly attached on said first flat surface of said flat portion, said vial carrier support means are fixedly attached on said second flat surface of said flat portion, said vial carrier finger thrust means are fixedly attached on said vial carrier support means.

12. A syringe handheld safety apparatus as set forth in claim 9 where said interchangeable struck limiter is slidably supported and selectively rigidly restrained on said interchangeable syringe carrier includes a syringe plugger stop limit portion attached fixedly on a support portion, said support portion includes engraved markings corresponding to a particular struck length reflecting said syringe carrier struck limiter restraining means.
